(19) United States
(12) Patent Application Publication
(10) Pub. No.: US 2005/0240180 A1
(43) Pub. Date: Oct. 27, 2005

(54) Spinal osteosynthesis system comprising a support pad

(76) Inventors: Cecile Vienney, Bordeaux (FR); Cedric De Coninck, Cestas Gazinet (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/488,633

(22) PCT Filed: Sep. 3, 2002
(86) PCT No.: PCT/FR02/02998

§ 371 Date: Oct. 19, 2004

(30) Foreign Application Priority Data
Sep. 3, 2001 (FR) 01/11363

(51) Int. Cl. A61B 17/58

(52) U.S. Cl. 606/61

(57) ABSTRACT

The invention concerns a spinal osteosynthesis system comprising an anchoring member (1), a linking member (2), a lock (4) and interposition means (3) adapted to be placed in mounted position between the lock and the linking member when the latter are received in the anchoring member and designed not to overlap the anchoring member in mounted position, the interposition means (3) having at least one ridge for providing a support on the linking member.

The invention concerns clamping means intended for spinal osteosynthesis systems.

The document FR 2 680 461 presents a device comprising a bone anchoring member of the pedicle screw type comprising a U-shaped head able to receive firstly a bolt and secondly a ring surrounding the arms forming the U. In addition, the ring comprises a diametral bar able to come between the arms of the U-shaped opening and able to be interposed between the bolt and a connecting rod received between the arms of the U when the assembly is tightened. This device has drawbacks in terms of volume and complexity:
the ring is bulky and extends so as to project from the external surface of the head of the anchoring member whilst an osteosynthesis system must today be invasive to the minimum possible extent;
the ring being mounted so as to slide on the arms of the U, when the bolt is tightened, the form of the threads obliges the arms to move away from each other, blocking the descent of the ring, as well as the deformation of the diametral arm. The result is poor abutment of the diametral arm on the connecting rod, resulting in a high risk of unclamping of the osteosynthesis system which is prejudicial to the patient;
the bolt and the ring are independent of each other, making assembly complex and tedious.

One aim of the invention is to provide clamping means for a spinal osteosynthesis system making it possible to provide a repeatable and sufficient axial clamping force with a minimum clamping torque, whilst being simple and compact.

For this purpose there is provided, according to the invention, an osteosynthesis system comprising an anchoring member, a connecting member, a bolt and interposition means able to be placed in mounting position between the bolt and the connecting member when these are received in the anchoring member, these means being arranged so as to not project beyond the anchoring member in the-mounting position.

Thus the interposition means remain invasive to the minimum extent whilst providing a repeatable and sufficient axial locking force for a minimum clamping torque.

Advantageously, the spinal osteosynthesis system has at least one of the following characteristics:
the interposition means are arranged so that they are able to come into abutment on the connecting member along at least one generatrix of the connecting member;
the interposition means are arranged so that they are able to come into abutment on the connecting member along two parallel generatrices of the connecting member;
the interposition means have at least one ridge able to effect an abutment on the connecting member;
the interposition means have a flat surface perpendicular to an axis of rotation of the bolt able to come into abutment with the bolt;
the interposition means have a concave face and a face delimiting at least one ridge able to come into abutment against the connecting member;
the concave face is a portion of a cylindrical face, a mean diameter of which is less than a dimension of the connecting member;
the concave face is delimited by at least two ridges able to effect the abutment against the connecting member;
the system has means of holding the interposition means directly on the bolt;
the interposition means are able to be rotatably mounted on the bolt;
the holding means comprise a lip;
the holding means comprise at least one lug able to come into abutment on the lip;
the bolt comprises the lip and the interposition means comprise the lug;
the bolt comprises the lug and the interposition means comprise the lip;
the holding means comprise a thread able to be held captive by the lip;
the lip forms part of a groove;
the interposition means have a thickness e between their top and bottom faces able to make them substantially undeformable when the system is tightened.

There is thus provided according to the invention an osteosynthesis system for the spinal column comprising at least one system having at least one of the aforementioned characteristics.

Other characteristics and advantages of the invention will emerge during the following description of a preferred embodiment. In the accompanying drawings:

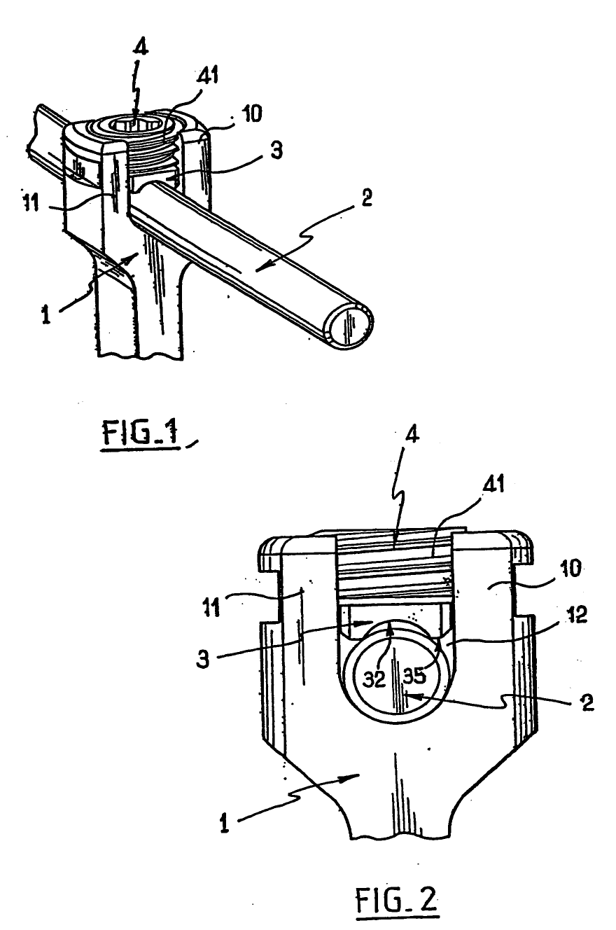

FIG. 1 is a partial view in three dimensions of an osteosynthesis system according to a preferred embodiment of the invention;

FIG. 2 is a side view of the osteosynthesis system of FIG. 1;

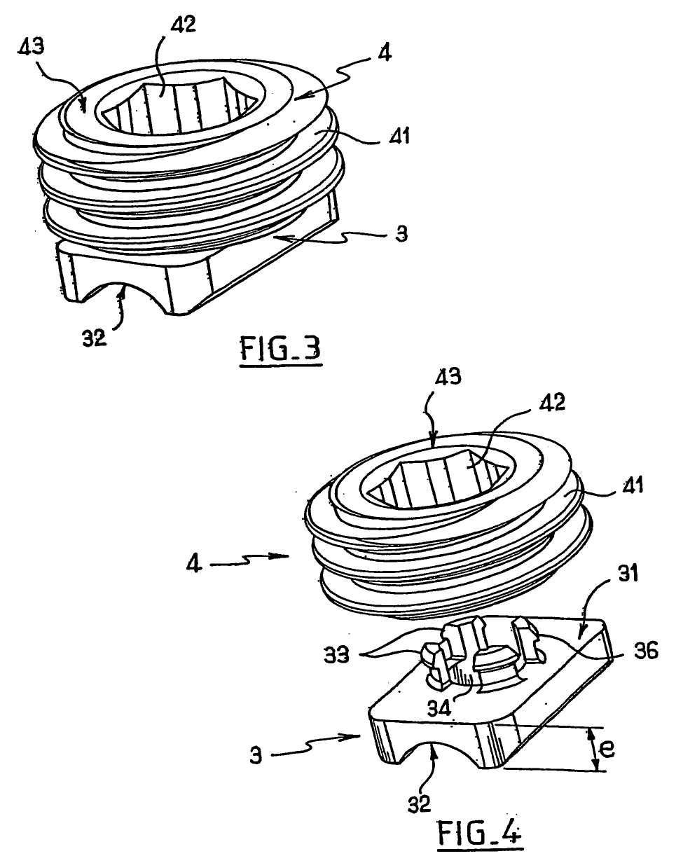

FIG. 3 is a view in three dimensions of the bolt and pad of the osteosynthesis system of FIG. 1;

FIG. 4 is an exploded view in three dimensions of the pieces in FIG. 3;

FIG. 5 is a view in three dimensions and a cross-section through the pieces in FIG. 3;

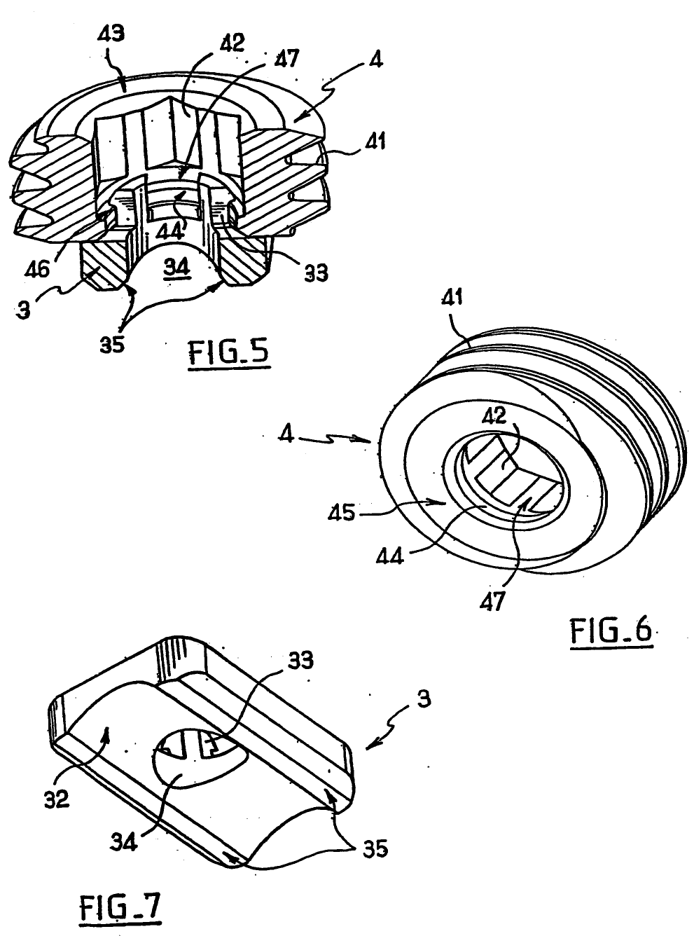

FIG. 6 is a view in three dimensions of the bolt in FIG. 3; and

FIG. 7 is a view in three dimensions of the pad in FIG. 3.

With reference to FIGS. 1 and 2, the osteosynthesis system comprises several sub-assemblies each comprising an anchoring member 1 having a head able to receive a connecting member 2 as well as a bolt 4 and a clamping pad 3. One of these sub-assemblies is illustrated in FIG. 1.

The anchoring member 1 has a head comprising two arms 11 and 12 extending opposite each other in order to form a U-shaped opening 12. The connecting member 2, here a connecting rod, is received in the U-shaped opening 12 of the anchoring member 1. The internal faces facing each other of the arms 11 and 12 of the head of the anchoring member comprise a third able to cooperate with a thread 41 of the bolt 4. The pad 3 can be positioned between the anchoring member 2 and the bolt 4.

The bolt 4 has the general form of a cylinder. Its lateral external face has a thread 41 able to cooperate with the complementary thread on the anchoring member 1. The bolt 4 has a top face 43 perpendicular to its axis of revolution (not shown). From this face 43, the member 4 has a central circular orifice 47 terminating in a lip 46 extending so as to project internally in a direction radial to the axis. In the major part of the orifice 47, means 42 of using the bolt 4 are produced and extend from the top face 43, over the major part of the orifice 47. From the edge 46 of the orifice 47, a through hole 44 with a circular cross-section is produced in the bolt 4 as far as a bottom face 45 parallel to the top face 43. The diameter of the hole 44 is less than the diameter of the orifice 47, which makes it possible to obtain the lip 46 between the hole 44 and the orifice 47.

The pad 3 is in the overall form of a rectangular parallelepiped. It has a width such that it can be inserted between the two arms 10 and 11 of the anchoring member forming the opening 12. The clamping pad 3 has a length such that, once inserted in the U-shaped opening 12, it does not project on either side of the head of the anchoring member 1 in the longitudinal direction of the connecting member 2. Preferentially, the length of the clamping pad 3 is substantially equal to the largest outside diameter of the bolt 4.

The clamping pad 3 has a flat top face 31 at the centre of which a through orifice 34 can be produced over the entire thickness of the clamping pad 3. Around this orifice there are disposed, uniformly distributed around this orifice 34, several lugs 33 extending so ad to project from the face 31 of the clamping pad 3. Here the lugs are four in number. Each of the lugs has, at its free end on its external face, a tooth 36. Moreover, each of the lugs 33 is deformable elastically in radial flexion with respect to the axis of the orifice. The clamping pad 3 has on its bottom face, opposite to the top face 31, a profiled concave face 32 with a circular cross-section and extending over the entire length of the clamping pad 3. On-each side of this concave face, the clamping pad 3 has, over its entire length, a flat face 35 perpendicular to an axis of the orifice 34 and parallel to the top face 31. The intersection of this flat face 35 with the concave face 32 determines a ridge which is able to come into abutment, during clamping, with the surface of the connecting member 2, as will be seen below. In variant embodiments, the face 35 can be principally concave or convex.

The clamping pad 3 is able to be mounted on the bolt 4, as illustrated in FIG. 5. For this purpose, the lugs 33 on the clamping pad 3 are inserted in the hole 44 in the bolt 4. At the time of this insertion, the lugs 33 deform towards the inside of the orifice 34, and this elastically. Once the teeth 36 of the lugs 33 emerge in the orifice 47, the lugs 33, because of their elasticity, return to their initial position, putting the teeth 36 on each of the lugs 33 in contact with the radial internal projection 46. Thus the clamping pad 3 is mounted captive on the bolt 4, whilst leaving the bolt 4 free to rotate with respect to the clamping pad 3.

During use, the surgeon, once he has positioned the connecting member 2 within the U-shaped opening in the anchoring member 1, introduces the assembly formed from the clamping pad and bolt 4 between the arms 10 and 11 of the anchoring member, the clamping pad 3 being directed towards the connecting member 2. When the surgeon rotates the bolt 4 so that the latter descends within the U-shaped opening, the clamping pad slides along the arms 10 and 11 of the anchoring member 1 pushed by the bolt 4 until they come into contact with the connecting member 2. The surgeon then clamps his assembly, the locking of the connecting member 2 being effected by contact thereof with the clamping pad via the ridges determined by the intersection of the concave face 32 and the flat faces 35 of it. Thus linear contact is effected along two generatrices of the connecting member 2. This is made possible by the fact that the mean radius of the section of the concave face 32 of the clamping pad 3 is less than the radius of the rod forming the connecting member 2. In addition, under the clamping pressure, the ridge will mate with the surface of the connecting member, providing additional security against unclamping of the assembly.

Naturally, many modifications can be made to the invention, without for all that departing from the scope thereof. For example:
the rim can be replaced by a groove;
the lugs 33 can be replaced by a thread which will cooperate with an equivalent thread provided in the hole 44, the thread on the clamping pad surmounting a groove able to receive the thread produced on the orifice 44. The latter will then be captive when the clamping pad is assembled with the bolt in this groove, the captive thread can be on the clamping pad and the groove can be on the bolt;
the concave face 32 can have a radius substantially equivalent to that of the connecting member 2 but will then have a multitude of ridges parallel to each other, uniformly distributed over the entire surface of this face, these ridges coming into engagement with the surface of the connecting member during clamping.

1. A spinal osteosynthesis system comprising an anchoring member (1), a connecting member (2), a bolt (4), and interposition means (3) able to be placed in mounting position between the bolt and the connecting member when the latter are received in the anchoring member and arranged so as not to project beyond the anchoring member in the mounting position, characterised in that the interposition means (3) have at least one ridge able to produce an abutment on the connecting member.

2. A system according to claim 1, characterised in that the interposition means (3) are arranged so that they are able to come into abutment on the connecting member along at least one generatrix of the connecting member.

3. A system according to claim 1 or 2, characterised in that the interposition means (3) are arranged so that they are able to come into abutment on the connecting member along two parallel generatrices of the connecting member.

4. A system according to one of claims 1 to 3, characterised in that the interposition means (3) have a flat surface (31) perpendicular to an axis of rotation of the bolt able to come into abutment against the bolt.

5. A system according to claims 1 to 4, characterised in that the interposition means (3) have a concave face (32) and a face (35) delimiting at least one ridge able to come into abutment against the connecting member.

6. A system according to claim 6, characterised in that the concave face is a portion of a cylindrical face, a mean diameter of which is less than a diameter of the connecting member.

7. A system according to claim 5 or 6, characterised in that the concave face is delimited by at least two ridges able to effect the abutment against the connecting member.

8. A system according to one of claims 1 to 7, characterised in that the interposition means (3) are able to be rotatably mounted on the bolt.

9. A system according to one of claims 1 to 8, characterised in that it has means (33, 46) of holding the interposition means (3) directly on the bolt.

10. A system according to claim 9, characterised in that the holding means (33, 46) comprise a lip (46).

11. A system according to claim 10, characterised in that the holding means (33, 46) comprise at least one lug (33) able to come into engagement with the lip (46).

12. A system according to claim 11, characterised in that the bolt comprises the lip and the interposition means (3) comprise the lug.

13. A system according to claim 12, characterised in that the bolt comprises the lug and the interposition means (3) comprise the lip.

14. A system according to claim 1, characterised in that the holding means (33, 46) comprise a thread able to be held captive by the lip.

15. A system according to one of claims 1 to 14, characterised in that the lip forms part of a groove.

16. A system according to one of claims 1 to 15, characterised in that the interposition means (3) have a thickness e between their top and bottom faces able to make them substantially undeformable when the system is tightened.
